(19) United States
(12) Patent Application Publication
(10) Pub. No.: US 2026/0266801 A1
(43) Pub. Date: Sep. 10, 2026

(54) SYSTEM FOR GENERATING HIGH-PRECISION GAS MIXTURES

(71) Applicant: FEDERAL INSTITUTE OF METROLOGY, Berne-Wabern (CH)

(72) Inventors: Bernhard NIEDERHAUSER, Berne-Wabern (CH); Martin STALDER, Berne-Wabern (CH); Daniel SCHWALLER, Berne-Wabern (CH); Kevin AUDERSET, Berne-Wabern (CH)

(21) Appl. No.: 19/491,370

(22) PCT Filed: Jul. 3, 2023
(86) PCT No.: PCT/IB2023/056895

§ 371 Date: Dec. 8, 2025

(51) Int. Cl. G01N 33/497; G01N 1/22; G01N 1/38

(52) U.S. Cl. CPC G01N 33/4972; G01N 1/22; G01N 1/38; G01N 2001/2244

(57) ABSTRACT

Embodiments of the present disclosure may comprise a system for generating high-precision gas mixtures, the system comprising a first part that may be temperature-controlled to a first temperature, comprising a first gas generator. Embodiments may also comprise one or more second gas generators. Embodiments may also comprise a second part that may be temperature-controlled to a second temperature and comprise a gas mixing assembly.

### TECHNICAL FIELD

The present disclosure generally relates to a system for generating high-precision gas mixtures.

### BACKGROUND

Aspects of the present disclosure relate to a system for generating high-precision gas mixtures. Various issues may exist with conventional solutions for a system for generating high-precision gas mixtures. In this regard, conventional systems and methods for a system for generating high-precision gas mixtures may be costly, cumbersome, and/or inefficient.

Limitations and disadvantages of conventional systems and methods will become apparent to one of skill in the art, through comparison of such approaches with some aspects of the present methods and systems set forth in the remainder of this disclosure with reference to the drawings.

### BRIEF SUMMARY OF THE DISCLOSURE

Shown in and/or described in connection with at least one of the figures, and set forth more completely in the claims are systems for generating high-precision gas mixtures.

These and other advantages, aspects and novel features of the present disclosure, as well as details of illustrated embodiments thereof, will be more fully understood from the following description and drawings.

### BRIEF DESCRIPTION OF THE FIGURES

The various features and advantages of the present disclosure may be more readily understood with reference to the following detailed description taken in conjunction with the accompanying drawings, wherein like reference numerals designate like structural elements.

FIG. 1 is a block diagram illustrating a system, according to some embodiments of the present disclosure.

FIG. 2 is a block diagram further illustrating the system from FIG. 1, according to some embodiments of the present disclosure.

FIG. 3A illustrates an exemplary diagram of the ethanol concentration of a temperature-regulated uncoated piping.

FIG. 3B illustrates an exemplary diagram of the ethanol concentration of a temperature-regulated coated piping.

FIG. 4 illustrates an exemplary gas mixing assembly.

### DETAILED DESCRIPTION

The following discussion provides various examples of embodiments of the disclosure. Such examples are non-limiting, and the scope of the appended claims should not be limited to the particular examples disclosed. In the following discussion, the terms “example” and “e.g.” are non-limiting.

The figures illustrate the general manner of construction, and descriptions and details of well-known features and techniques may be omitted to avoid unnecessarily obscuring the present disclosure. In addition, elements in the drawing figures are not necessarily drawn to scale. For example, the dimensions of some of the elements in the figures may be exaggerated relative to other elements to help improve understanding of the examples discussed in the present disclosure. The same reference numerals in different figures denote the same elements.

The term “or” means any one or more of the items in the list joined by “or”. As an example, “x or y” means any element of the three-element set {(x), (y), (x, y)}. As another example, “x, y, or z” means any element of the seven-element set {(x), (y), (z), (x, y), (x, z), (y, z), (x, y, z)}.

The terms “comprises,” “comprising,” “comprises,” and/or “including,” are “open ended” terms and specify the presence of stated features, but do not preclude the presence or addition of one or more other features.

The terms “first,” “second,” etc. may be used herein to describe various elements, and these elements should not be limited by these terms. These terms are only used to distinguish one element from another. Thus, for example, a first element discussed in this disclosure could be termed a second element without departing from the teachings of the present disclosure.

Unless specified otherwise, the term “coupled” may be used to describe two elements directly contacting each other or describe two elements indirectly connected by one or more other elements. For example, if element A is coupled to element B, then element A can be directly contacting element B or indirectly connected to element B by an intervening element C. Similarly, the terms “over” or “on” may be used to describe two elements directly contacting each other or describe two elements indirectly connected by one or more other elements.

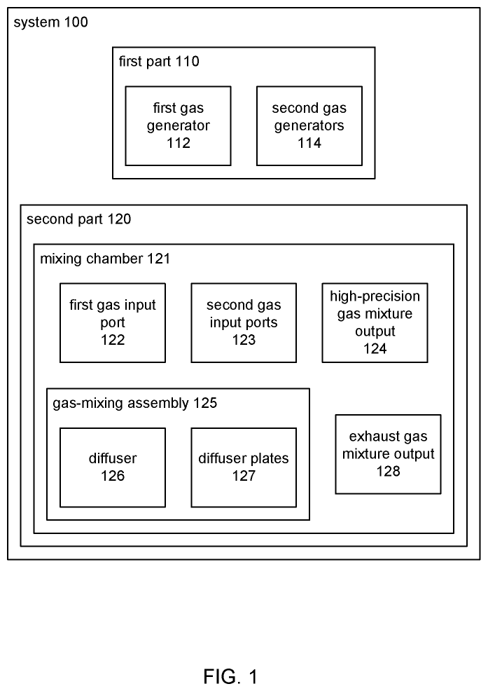

Referring now to FIG. 1, the system 100 may be operable to generate a high precision gas mixture. For example, such gas mixtures may be used to test evidential breath analyzers. Evidential breath analyzers may be devices used to measure alcohol/ethanol content in human breath. As will be obvious to the person skilled in the art, to test evidential breath analyzers, the test gas must be highly accurate to verify the analyzer measurements. For this reason, it may be desirable to generate a high precision gas that models a human breath comprising ethanol.

In certain prior art systems, a piston combined with a cylinder of appropriate volume (about 5 liters), may be used as an artificial lung. Because of its relatively large volume, such an artificial lung may act as an integrator that may average out temporary fluctuations in the gas compositions at its inputs. For example, if the ethanol concentration fluctuates in time, the large artificial lung may nevertheless comprise a correct homogeneous ethanol air mixture due to its integrative function and volume. One disadvantage of such prior art systems is however the large volume required of the artificial lung, leading to a large size test apparatus. In addition, when mechanically emptying the artificial lung by moving the piston in the cylinder, some residual gas may remain in the cylinder. Thus, when the gas mixture in the artificial lung needs to be adjusted, it may typically be necessary to go through several fill-empty cycles of the artificial lung to ensure that all residual gas has been expelled. Correspondingly, one disadvantage of prior art systems may be the large physical size and the slow adaptability because of the gas expelling necessary.

FIG. 1 is a block diagram that describes a system 100, according to some embodiments of the present disclosure. In some embodiments, the system 100 may comprise a first part 110 that may be temperature-controlled to a first temperature and a second part 120 that may be temperature-controlled to a second temperature. The first part 110 may comprise a first gas generator 112 and one or more second gas generators 114. The second part 120 may comprise a mixing chamber 121. The second temperature may be higher than the first temperature.

In some embodiments, the mixing chamber 121 may comprise a high-precision gas mixture output 124 and an exhaust gas mixture output 128. The mixing chamber 121 may also comprise a first gas input port 122, the first gas input port 122 coupled to the first gas generator 112 by coated piping. The mixing chamber 121 may also comprise one or more second gas input ports 123, each coupled to one of the one or more second gas generators 114 by piping. The mixing chamber 121 may also comprise a gas-mixing assembly 125. The mixing chamber 121 may be temperature-controlled to the second temperature. The coated piping between the first gas generator 112 and the mixing chamber 121 may be temperature controlled to the first temperature. The piping between the second gas generators 114 and the mixing chamber 121 may also be temperature-controlled to the first temperature. The gas-mixing assembly 125 may comprise a diffuser 126 coupled to the first gas input by coated piping, and one or more diffuser plates 127.

In some embodiments, the system 100 may be an evidential breath analyzer test system. In some embodiments, the high precision gas mixture may be modeling a human breath. In some embodiments, gas generated by the first gas generators and/or the one or more second gas generators 114 may be air substantially saturated by a chemical compound vapor. In some embodiments, a gas generated by the first gas generator 112 may be air substantially saturated by ethanol vapor at the first temperature.

In some embodiments, substantially saturated air may be air 92-100% saturated with ethanol vapor. In some embodiments, a gas generated by the one or more second gas generators 114 may be a gas mixture of air and carbon dioxide, 92-100% saturated with water vapor. In some embodiments, the mixture of air and carbon dioxide may be preheated to the first temperature before water vapor may be added.

In some embodiments, the second temperature may be chosen sufficiently higher than the first temperature to avoid condensation of gases generated by the first gas generator 112 and the one or more second gas generators 114 in the mixing chamber 121. In some embodiments, the coating of the coated piping may be operable to prevent adsorption of a gas generated in the first gas generator 112. In some embodiments, the mixing chamber 121 may be coated with a same or similar coating to that used for the coated piping.

In some embodiments, the coating may be an inert non-reactive silicon coating. In some embodiments, the coating may be applied by a chemical vapor deposition process to the coated piping and/or the mixing chamber 121. In some embodiments, the diffuser 126 may be made from a sintered material. In some embodiments, the one or more diffuser plates 127 may comprise a plurality of holes. In some embodiments, a blower may be coupled to the high-precision gas mixture output 124.

In some embodiments, the blower may extract the high-precision gas mixture from the mixing chamber 121 and feed it to a device under test. In some embodiments, the exhaust gas mixture output 128 may be used to discharge a part of the high-precision gas mixture from the mixing chamber 121, so that the gas flow from the mixing chamber 121 into the high-precision gas mixture output 124 plus the exhaust gas mixture output 128 may be approximately constant over some unit of time.

In some embodiments, the high-precision gas mixture may comprise air, carbon dioxide, water vapor, and ethanol.

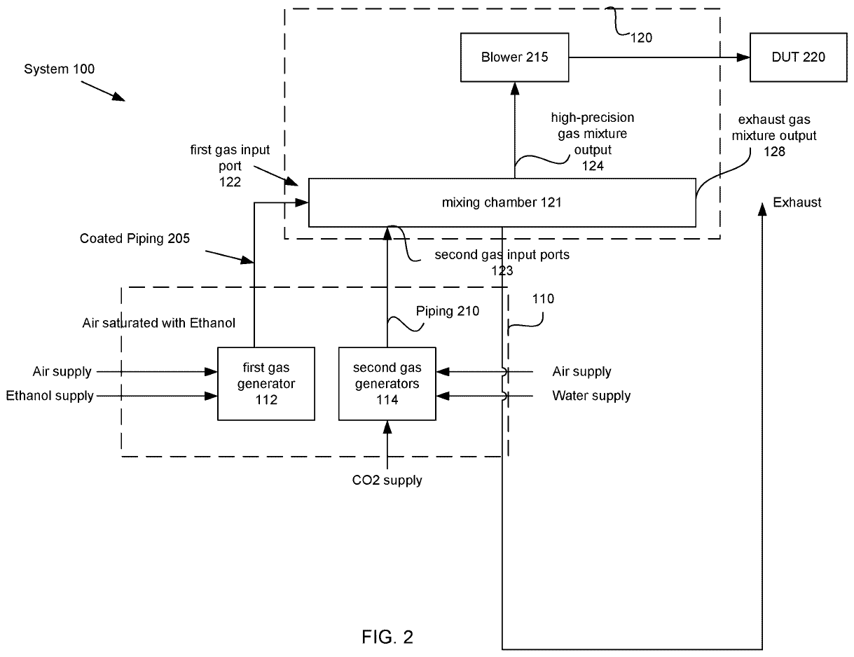

FIG. 2 illustrates an exemplary embodiment of a system 100 for generating high-precision gas mixtures. Similar reference numbers as in FIG. 1 refer to similar elements.

The first gas generator 112 may be operable to generate a gas mixture comprising air saturated with ethanol vapor at its output from an air supply input and an ethanol supply input. In accordance with various embodiments of the disclosure, saturated may refer to 92% to 100% ethanol vapor saturation of the gas mixture at the output of the first gas generator 112. The gas flow at the output of the first gas generator 112 may be relatively small when used to model human breath comprising ethanol. For example, in accordance with various embodiments of the disclosure, the gas flow at the output of the first gas generator 112 may be less than 100 milliliters per minute. The output of the first gas generator 112 may be coupled to a first gas input port 122 of the mixing chamber 121 using coated piping 205. The input port 122 and the piping 205 may be coated because the small quantity gas flow from the first gas generator 112 may be partly adsorbed by the piping 205 otherwise. When a part of the air comprising ethanol is adsorbed by the piping 205, the ethanol vapor content of the gas transported in piping 205 may vary, which may be undesirable. Accordingly, it is beneficial to use anti-adsorption coating for the coated piping 205. As the quantity of ethanol vapor that may be at the output of the first gas generator 112 may be temperature-dependent, a first part 110 of the system 100 may be temperature-controlled. For example, the first part 110 of the system 100 may be maintained at a first temperature of 33.7° C. To ensure that the gas at the output of the first gas generator 112 may not condense onto the walls of the coated piping 205, the coated piping 205 may be temperature-controlled at the first temperature 33.7° C., either additionally or as part of the first part 110 of the system 100.

Similarly, one or more second gas generators 114 (only one exemplary second gas generator 114 is shown in FIG. 2) may be used to generate a gas mixture comprising air, carbon dioxide (CO2), and water vapor, for example. For example, the air and carbon dioxide mixture may be saturated with water vapor. In accordance with various embodiments of the disclosure, saturated may refer to 92% to 100% water vapor saturation of the gas mixture at the output of the second gas generator 114. In accordance with various embodiments of the disclosure, the air and carbon dioxide may be mixed and preheated to a first temperature of 33.7° C. before being enriched with water vapor. In some instances, it may also be possible to feed the preheated air and carbon dioxide mixture into the mixing chamber 121 dry, i.e., without added water vapor and thus unsaturated. Generally, it may be desirable that the output gas flow of the second gas generator 114 may be significantly larger than the output gas flow of the first gas generator 112. For example, the output gas flow of the second gas generator 114 may be 10 to 50 liters per minute. The output of the second gas generator 114 may be coupled to a second gas input port 123 of the mixing chamber 121 via piping 210. The piping 210 may be stainless steel, for example. As for the first gas generator 112, the saturation of the output gas mixture of the second gas generator 114 may be temperature-dependent. Correspondingly, the second gas generator 114 may be comprised in a first part 110 of the system 100 that may be temperature controlled. The piping 210 may be temperature controlled to a first temperature, for example, 33.7° C., either additionally or as part of the first part 110 of the system 100.

The mixing chamber 121 may be operable to generate a homogeneous gas mixture available at its outputs 124, 128 from its input gases at a first gas input port 122 and a second gas input port 123. In accordance with various embodiments of the disclosure, there may be one or more second gas input ports 123. To avoid adsorption of the air-ethanol gas mixture entering the mixing chamber 121 from the first gas input port 122, the mixing chamber 121 and the input port 122 may be coated in an anti-adsorption coating, similar to coated piping 205. To achieve a homogeneous gas mixture within the mixing chamber 121, the mixing chamber 121 may comprise a gas mixing assembly 125. Furthermore, to ensure that the gases entering via the input ports 122, 123 may not condense on the walls of the mixing chamber 121, the mixing chamber 121 and the second part 120 of the system 100 may be temperature-controlled to a second temperature. The second temperature of a second part 120 may be chosen such that it is higher than the first temperature of a first part 110. For example, a second temperature may be 35° C. The mixing chamber 121 may be much smaller than a prior art artificial lung, for example 100 to 500 milliliters. Such a volume for the mixing chamber 121 may be possible because of the coated piping 205, the temperature control of the first part 110 and the second part 120, and/or the coating of the mixing chamber 121. These features alone and/or in combination may ensure that the gas mixtures entering the mixing chamber 121 may be highly stable and highly accurate. The high precision gas mixture may then be output via output port 124 from the mixing chamber 121 to a device under test (DUT) 220 via a blower 215. The blower 215 may be adjustable and may provide a desirable gas flow to the DUT 220. For example, the blower 215 may be operable to generate a gas flow modeling a human breath/expiration comprising ethanol.

Because e.g., the volume of the mixing chamber 121 is smaller than the volume of prior art artificial lungs, the mixing chamber 121 may be suitable for faster adaptation of gas mixture compositions.

In accordance with various embodiments of the disclosure, it may be advantageous for the gas mixture obtained in the mixing chamber 121 to maintain a constant gas flow through the mixing chamber 121 for a certain time. In such a case, because the gas flow at the high precision gas mixture output port 124 may be variable to model a human breath for the DUT 220, the remainder of the gas from mixing chamber 121 may be expelled at the exhaust gas mixture output 128.

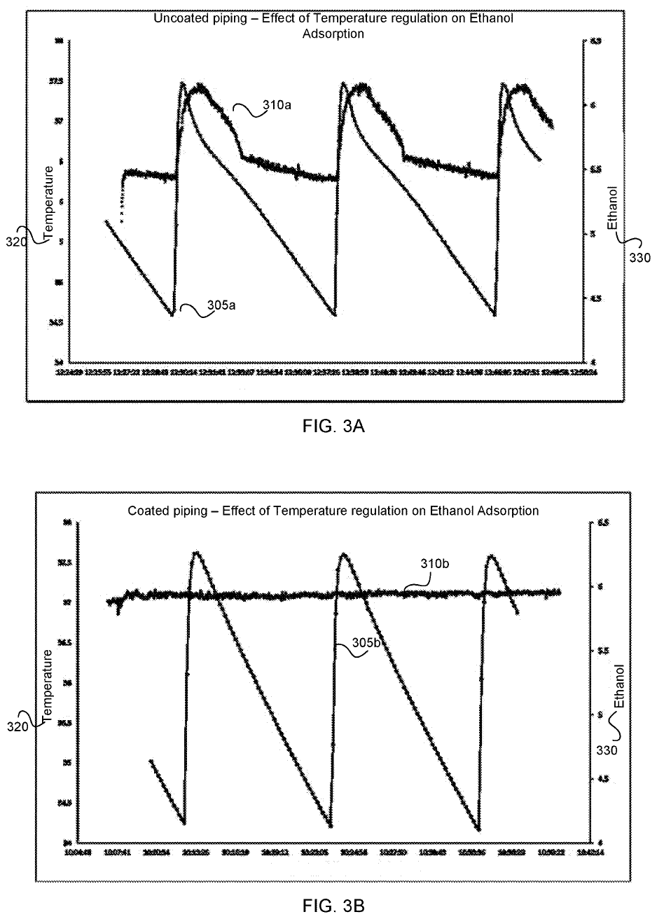

FIG. 3A illustrates an exemplary diagram of the ethanol concentration of a temperature-regulated uncoated piping 205. The x-axis shows time. The y-axis on the left 320 shows piping 205 temperature in degree Celsius, associated with the temperature of the piping 205 shown by temperature 305a. The y-axis on the right 330 shows an ethanol content in the gas flowing through piping 205. It may be seen how the ethanol content 310a in the gas through uncoated piping 205 may vary due to temperature variations. Specifically, as the temperature 305a may vary between 34.5 and 37.5° C. because of the temperature regulation of the piping 205, the adsorption of the piping 205 may vary too, and thus the ethanol content of the gas 310a may vary also. In practice, the temperature variation due to the temperature control in the piping 205 may be much smaller than illustrated but adsorption of the piping 205 may still be affected but to a lesser degree.

FIG. 3B illustrates an exemplary diagram of the ethanol concentration of a temperature regulated coated piping 205. The x-axis shows time. The y-axis on the left 320 shows coated piping 205 temperature in degree Celsius, associated with the temperature of the coated piping 205 shown by temperature 305b. The y-axis on the right 330 shows an ethanol content in the gas flowing through piping 205. It may be seen how the ethanol content 310b in the gas through coated piping 205 varies very little due to temperature variations in the piping. Correspondingly, FIG. 3B illustrates that the ethanol content 310b may remain much more stable over varying temperatures 305b for a coated piping 205, compared to uncoated piping 205 shown in FIG. 3A. The coating may be advantageously selected to have anti-adsorption properties for ethanol or another chemical compound, as generated in the first gas generator 112.

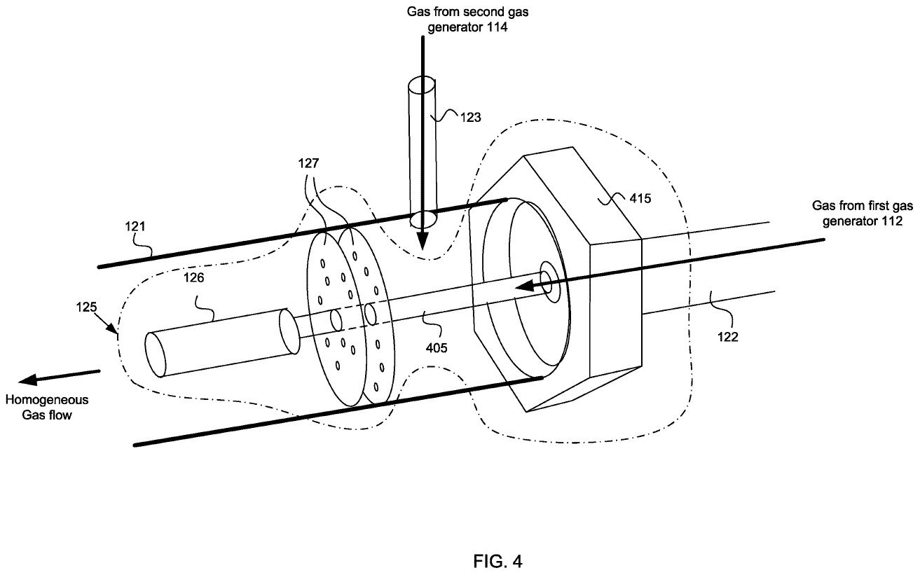

FIG. 4 illustrates an exemplary gas mixing assembly 125. There is shown a gas mixing assembly 125 comprising a diffuser 126, diffuser plates 127, and a coated piping 405. There is further shown a first gas input port 122, an attachment nut 415, the mixing chamber 121, and a second gas input port 123. There are arrows showing gas flows from a first gas generator 112, the second gas generator 114, and a homogeneous gas flow. The attachment nut 415 may be enabled to couple the gas mixing assembly 125 to the mixing chamber 121. The coated piping 405 is operable to couple the first gas input port 122 to the diffuser 126.

The gas mixing assembly 125 may be operable to generate a homogeneous gas mixture from the gas inputs from a first gas generator 112 and from a second gas generator 114. The gas from the first gas generator 112 may enter the gas mixing assembly 125 at a first gas input port 122. From there, the gas continues through further coated piping 405 to diffuser 126. The diffuser 126 may be made from a sintered material and the gas entering the diffuser 126 may escape through the diffuser 126 in a turbulent manner into the mixing chamber 121. The diffuser is formed by compacting and sintering metal particles to create a porous structure with interconnected voids. This sintered metal structure allows for the controlled passage and dispersion of gases or fluids, thereby enabling uniform distribution and diffusion of substances. The diffuser's sintered metal composition provides enhanced durability, resistance to high temperatures and corrosion, making it suitable for various applications in industries such as automotive, aerospace, and chemical processing.

The gas from the second gas generator 114 enters the mixing chamber behind the one or more diffuser plates 127. The gas from the second gas generator 114 traverses the one or more diffuser plates 127. The diffuser plates 127 may be, for example, metallic plates comprising holes. As the gas from the second gas generator 114 traverses the one or more diffuser plates 127 in the direction of the homogeneous gas flow, the gas from the second gas generator 114 is made turbulent. The turbulent gas from the second gas generator 114 after the diffuser plates 127 may thus efficiently mix with the turbulent gas from the first gas generator 112 exiting the diffuser 126. Typically, the gas from the second gas generator 114 may be a carbon dioxide, air, and water vapor mixture. Also, the flow of gas from the second gas generator 114 may typically be much larger than the gas flow from the first gas generator 112, for example 10 to 50 liters per minute. The gas flow from the first gas generator 112 may be, for example, less than 5 to 100 milliliters per minute. Due to the turbulent and strong gas flow after the diffuser plates 127, the turbulent gas from the first gas generator 112 exiting the diffuser 126 may rapidly generate a homogeneous gas mixture in the mixing chamber 121. The homogeneous gas mixture resulting may then be extracted/expelled via a high precision gas mixture output 124 and/or an exhaust gas mixture output 128 (not illustrated in FIG. 4).

The present disclosure comprises reference to certain examples, however, it will be understood by those skilled in the art that various changes may be made and equivalents may be substituted without departing from the scope of the disclosure. In addition, modifications may be made to the disclosed examples without departing from the scope of the present disclosure. Therefore, it is intended that the present disclosure not be limited to the examples disclosed, but that the disclosure will comprise all examples falling within the scope of the appended claims.

1. A system for generating high-precision gas mixtures, the system comprising:
a first part that is temperature-controlled to a first temperature, comprising:
a first gas generator; and
one or more second gas generators;
a second part that is temperature-controlled to a second temperature, wherein said second temperature is higher than said first temperature, comprising:
a mixing chamber, wherein said mixing chamber is temperature-controlled to said second temperature, further comprising:
a first gas input port said first gas input port coupled to the output of said first gas generator by coated piping, wherein said coated piping is temperature-controlled to said first temperature;
one or more second gas input ports, each coupled to the output of one of said one or more second gas generators by piping, wherein said piping is temperature-controlled to said first temperature;
a gas-mixing assembly said gas mixing assembly comprising:
a diffuser coupled to said first gas input port by coated piping; and
one or more diffuser plates;
a high-precision gas mixture output; and
an exhaust gas mixture output.

2. The system of claim 1, wherein said system is an evidential breath analyzer test system.

3. The system of claim 1, wherein the system is configured to generate, in use, said high precision gas mixture that is modeling a human breath comprising ethanol.

4. The system according to claim 1, wherein the system is configured to generate, in use, said high-precision gas mixture that comprises air, carbon dioxide, water vapor and ethanol.

5. The system according to claim 1, wherein, in use, a gas generated by said first gas generator and/or said one or more second gas generators is air substantially saturated by a chemical compound vapor.

6. The system according to claim 1, wherein, in use, a gas generated by said first gas generator is air substantially saturated by ethanol vapor at said first temperature.

7. The system according to claim 1, wherein, in use, a gas generated by said one or more second gas generators comprises a mixture of air and carbon dioxide, substantially saturated by water vapor at said first temperature.

8. The system according to claim 1, wherein, in use, said second temperature is chosen sufficiently higher than said first temperature to avoid condensation of gases generated by said first gas generator and said one or more second gas generators in said mixing chamber.

9. The system according to claim 1, wherein said coating of said coated piping is operable to prevent adsorption of a gas generated in said first gas generator.

10. The system of claim 9, wherein said mixing chamber is coated with said coating.

11. The system of claim 9, wherein said coating is an inert non-reactive silicon coating.

12. The system according to claim 9, wherein said coating is applied by a chemical vapor deposition process to said piping for coating.

13. The system according to claim 1, wherein said diffuser is made from a sintered material.

14. The system according to claim 1, wherein said diffuser plates comprise a plurality of holes.

15. The system of claim 6, wherein substantially saturated comprises said air 92-100% saturated with ethanol vapor.

16. The system of claim 7, wherein substantially saturated comprises said mixture of air and carbon dioxide 92-100% saturated with water vapor.

17. The system of claim 7, wherein said mixture of air and carbon dioxide is preheated to said first temperature before water vapor is added.

18. The system according to claim 1, wherein a blower is coupled to said high-precision gas mixture output.

19. The system of claim 18, wherein said blower is configured to extract said high-precision gas mixture from said mixing chamber and to feed it to a device under test.

20. The system according to claim 1, wherein said exhaust gas mixture output is used to discharge a part of said high-precision gas mixture from said mixing chamber, so that the gas flow from said mixing chamber into said high-precision gas mixture output plus said exhaust gas mixture output is approximately constant over some unit of time.
